(19) United States
(12) Patent Application Publication
(10) Pub. No.: US 2025/0066425 A1
(43) Pub. Date: Feb. 27, 2025

(54) POLYPEPTIDE AND APPLICATION THEREOF IN PREPARATION OF ANTI-FIBROSIS DRUG

(71) Applicant: Zhejiang University, Zhejiang (CN)

(72) Inventors: Xinyang HU, Zhejiang (CN); Jianan WANG, Zhejiang (CN); Cheng NI, Zhejiang (CN); Lingjun WANG, Zhejiang (CN); Shuchang YE, Zhejiang (CN)

(73) Assignee: Zhejiang University, Zhejiang (CN)

(21) Appl. No.: 18/726,124

(22) PCT Filed: Oct. 31, 2023
(86) PCT No.: PCT/CN2023/128247

§ 371 Date: Jul. 2, 2024

(30) Foreign Application Priority Data
Nov. 23, 2022 (CN) 202211480053.9
Apr. 7, 2023 (CN) 202310368198.8

(51) Int. Cl. C07K 14/00; A61K 9/00; A61K 38/00; A61P 9/10; A61P 11/00; A61P 13/12; A61P 43/00

(52) U.S. Cl. CPC C07K 14/001; A61K 9/0019; A61P 9/10; A61P 11/00; A61P 13/12; A61P 43/00; A61K 38/00

(57) ABSTRACT

The present invention relates to a polypeptide and application thereof in preparation of an anti-fibrosis drug. The polypeptide of the present invention has an amino acid sequence as shown in SEQ ID NO. 1. The polypeptide of the present invention has good cell penetrability, can efficiently enter target cells, can permanently exist in a target organ after reaching the target organ, and has no obvious toxic or side effect in an effective dose range; can effectively inhibit a classic signaling pathway of fibrosis, thereby inhibiting expression of proteins, such as fibrosis markers and extracellular matrices; and can effectively inhibit fibrosis of a target organ and improve functions of the target organ. A new drug and a solution are provided for clinical treatment of fibrosis diseases, and the polypeptide has important significance for improving a clinical treatment effect of the fibrosis diseases and improving life quality of patients.

### TECHNICAL FIELD

The present invention relates to the field of biomedicine, and in particular to a polypeptide and application thereof in preparation of an anti-fibrosis drug.

### BACKGROUND OF RELATED ART

Organ fibrosis diseases are characterized by excessive activation of interstitial cells and

excessive deposition of extracellular matrices. A variety of organs will have fibrosis diseases, including the heart, lung, kidney, skin, liver, bone marrow and the like. The fibrosis diseases, such as cardiac fibrosis, pulmonary fibrosis, renal fibrosis, skin fibrosis, liver cirrhosis and myelofibrosis, have caused a huge burden on patients.

Among them, cardiovascular diseases are the leading cause of death in the world. A

variety of cardiovascular diseases, such as coronary heart disease, hypertension, cardiomyopathy, arrhythmia and rheumatic heart disease, will lead to cardiac fibrosis, and the progression of cardiac fibrosis will further lead to aggravation of the cardiovascular diseases. Despite the prevalence, effective therapies for inhibiting or reversing the cardiac fibrosis are still lacking at present. In contrast, two drugs for treatment of idiopathic pulmonary fibrosis have been sold on the market, in which pirfenidone also has good efficacy in renal interstitial fibrosis and hepatic fibrosis.

Among the cardiovascular diseases, ischemic heart disease is a biggest killer, which accounts for 16% of the total number of deaths in the world. However, effective drugs for treatment of the cardiac fibrosis caused by the ischemic heart disease are still lacking. Therefore, research and development of drugs for treatment of the cardiac fibrosis have good social and economic benefits and a good development prospect.

Continuous abnormal activation of myofibroblasts mediated by various signals, such as a transforming growth factor b (TGFb), a platelet-derived growth factor and a fibroblast growth factor, has been considered as a major event in occurrence and progression of fibrosis. Among them, a TGFb pathway is one of the most widely studied pathways with the most significant effects, where a phosphorylated Smad2/Smad3 molecule is the most classic component in the pathway. However, only a few drugs, such as pirfenidone, targeting the TGFb pathway have been sold on the market at present, and more drugs targeting the pathway are required to be developed urgently. Therefore, research of anti-fibrosis drugs targeting the pathway has a great prospect.

On the other hand, polypeptides are natural active substances formed by covalently linking two or more amino acids with peptide bonds, which are widely found in nature and living bodies and play an important role in a life process. Polypeptide drugs have many characteristics, such as lower dose, higher selectivity, better specificity, better effect, less side effect, easiness in synthesis and customization and low cost. The polypeptide drugs have been widely concerned in drug development, and have been extensively studied in vitro, in vivo and at different stages of clinical treatment. However, due to the problems of molecular size, polarity, hydrophilicity, chargeability and the like, the polypeptides have difficulty in penetrating cell membranes, passing through physiological barriers, or passing through blood-brain barriers, like small molecules. Therefore, development of a polypeptide drug having not only a function of inhibiting fibrosis but also excellent cell penetrability has positive significance.

### SUMMARY OF THE INVENTION

In order to solve the above technical problems, the present invention provides a polypeptide and application thereof in preparation of an anti-fibrosis drug. The polypeptide of the present invention has good cell penetrability, can efficiently enter target cells, can permanently exist in a target organ after reaching the target organ, and has no obvious toxic or side effect in an effective dose range; can effectively inhibit a classic signaling pathway of fibrosis, thereby inhibiting expression of proteins, such as fibrosis markers and extracellular matrices; and can effectively inhibit fibrosis of a target organ and improve functions of the target organ.

The present invention has the following specific technical solutions.

In a first aspect, the present invention provides a polypeptide, named as PAFRK29, where PAF is an acronym of Peptide Anti-Fibrotic, and RK represents an abbreviation R of a first amino acid residue at an N terminal of the polypeptide and an abbreviation K of a last amino acid residue at a C terminal, respectively. An amino acid sequence is as shown in SEQ ID NO. 1.

Firstly, the polypeptide PAFRK29 of the present invention has a function of inhibiting fibrosis. Specifically, the polypeptide can inhibit a classic signaling pathway Smad2/3 of fibrosis, thereby inhibiting expression of a fibrosis marker protein aSMA and extracellular matrix proteins, such as Periostin, Fibronectin and Collagen 1a1; and can effectively inhibit fibrosis of a target organ and improve functions of the target organ.

Secondly, compared with most polypeptides, the polypeptide of the present invention has better cell penetrability, can efficiently enter target cells, can permanently exist in a target organ after reaching the target organ, and has no obvious toxic or side effect in an effective dose range. Therefore, the polypeptide of the present invention can be absorbed in vivo through a variety of administration methods (conventional polypeptides are not easily absorbed, which resulting in no efficacy).

In a second aspect, the present invention provides application of the polypeptide in

preparation of an anti-fibrosis drug.

As a preference, the fibrosis is cardiac fibrosis, pulmonary fibrosis, renal fibrosis, skin fibrosis, liver cirrhosis, or myelofibrosis.

Further, the cardiac fibrosis is cardiac fibrosis caused by ischemic heart disease, hypertensive heart disease, cardiomyopathy, arrhythmia, or rheumatic heart disease.

In a third aspect, the present invention provides an anti-fibrosis drug, including: a polypeptide PAFRK29, and a pharmaceutically acceptable carrier and/or an excipient.

As a preference, the anti-fibrosis drug is a pharmaceutical preparation administered by injection, oral administration, intranasal administration, pulmonary administration, rectal administration, oral mucosal administration, or skin administration.

Further, the injection includes intramyocardial injection, intradermal injection, subcutaneous injection, intramuscular injection, and intravenous injection.

Compared with the prior art, the present invention has the following beneficial effects:

(1) The polypeptide of the present invention has good cell penetrability, can efficiently enter target cells, can permanently exist in a target organ after reaching the target organ, and has no obvious toxic or side effect in an effective dose range; can effectively inhibit a classic signaling pathway of fibrosis, thereby inhibiting expression of proteins, such as fibrosis markers and extracellular matrices; and can effectively inhibit fibrosis of a target organ and improve functions of the target organ.

(2) According to the present invention, a new drug and a solution are provided for clinical treatment of fibrosis diseases, and the polypeptide has important significance for improving a clinical treatment effect of the fibrosis diseases and improving life quality of patients.

### BRIEF DESCRIPTION OF THE DRAWINGS

FIG. 1 shows Top15 polypeptides that are screened from a polypeptide library and have strong binding ability to a structural domain MH2 of SMAD3.

FIG. 2 shows HPLC purification results of a polypeptide PAFRK29.

FIG. 3 shows MS identification results of the polypeptide PAFRK29.

FIG. 4 shows enrichment images of the polypeptide PAFRK29 with a fluorescent marker FITC in primary neonatal rat cardiac fibroblasts, in which FIG.(a) is a fluorescence image of the polypeptide PAFRK29 without the fluorescent marker FITC, and FIG.(b) is a fluorescence image of the polypeptide PAFRK29 with the fluorescent marker FITC.

FIG. 5 shows an inhibitory effect of the polypeptide PAFRK29 on expression of fibrosis marker proteins and extracellular matrix proteins, such as aSMA, Periostin, Fibronectin and Collagen lal, in a fibrosis cell model.

FIG. 6 shows an inhibitory effect of the polypeptide PAFRK29 on a classic signaling pathway Smad2/3 of fibrosis in a fibrosis cell model.

FIG. 7 shows retension situation of the polypeptide PAFRK29 in hearts of healthy mice after intramyocardial injection of the polypeptide PAFRK29 with a fluorescent marker Cy3 at 1 ug at different time points.

FIG. 8 shows improvement situation of cardiac fibrosis and cardiac functions of mice with myocardial infarction after treatment with intramyocardial injection of the polypeptide PAFRK29 for several times, in which FIG.(a) shows representative Sirius Red staining images of general hearts and hearts after injection of the polypeptide PAFRK29, FIG.(b) shows changes in the proportion of a cardiac fibrosis area after injection of the polypeptide PAFRK29, and FIG.(c) shows improvement situation of a cardiac systolic function after injection of the polypeptide PAFRK29.

FIG. 9 shows retension situation of the polypeptide PAFRK29 in lungs and kidneys of healthy mice after tail vein injection of the polypeptide PAFRK29 with a fluorescent marker Cy3 at 10 mg/kg at different time points.

FIG. 10 shows improvement situation of renal fibrosis of renal fibrosis modeling mice (with unilateral ureteral obstruction) after treatment with tail vein injection of the polypeptide PAFRK29 for several times, in which FIG.(a) shows representative Masson staining images of paraffin sections of a kidney and a control kidney after injection of the polypeptide PAFRK29, and FIG.(b) shows statistical results of changes in the proportion of a fibrosis area in the kidney and the control kidney after injection of the polypeptide PAFRK29.

FIG. 11 shows improvement situation of pulmonary fibrosis of pulmonary fibrosis modeling mice (with intratracheal injection of bleomycin) after treatment with tail vein injection of the polypeptide PAFRK29 for several times, in which FIG.(a) shows representative Masson staining images of paraffin sections of a lung and a control lung after injection of the polypeptide PAFRK29, and FIG.(b) shows statistical results of changes in the proportion of a fibrosis area in the lung and the control lung after injection of the polypeptide PAFRK29.

FIG. 12 shows evaluation of a toxic or side effect of the polypeptide PAFRK29 on mice, in which FIG.(a) shows effect on blood urea nitrogen (BUN), FIG.(b) shows effect on activity of serum alanine aminotransferase (ALT), and FIG.(c) shows effect on activity of serum aspartate aminotransferase (AST).

### DETAILED DESCRIPTION OF THE EMBODIMENTS

The present invention is further described below in conjunction with specific examples. Terms used in the examples of the present invention are intended to describe specific embodiments, rather than to limit the scope of protection of the present invention. Without deviating from the spirit and scope of the concept of the present invention, all variations and advantages that can be thought of by persons skilled in the art are included in the present invention, and the scope of protection of the present invention is defined by the attached claims and any equivalents thereof. Experimental methods used in the following examples, unless otherwise specified, are conventional methods; and reagents and biological materials, unless otherwise specified, are commercially available.

### General Example

A polypeptide PAFRK29 has an amino acid sequence as shown in SEQ ID NO. 1 (RKKRRQRRRFQGQYFHSRQYKHPVGYEGK).

An anti-fibrosis drug includes: the polypeptide of claim 1 and a pharmaceutically acceptable carrier and/or an excipient.

As a preference, the fibrosis is cardiac fibrosis, pulmonary fibrosis, renal fibrosis, skin fibrosis, liver cirrhosis, or myelofibrosis. The anti-fibrosis drug is a pharmaceutical preparation administered by injection, oral administration, intranasal administration, pulmonary administration, rectal administration, oral mucosal administration, or skin administration. Further, the injection includes intramyocardial injection, intradermal injection, subcutaneous injection, intramuscular injection, and intravenous injection.

### Example 1: Screening and Optimization of Polypeptides

In the present invention, the polypeptide PAFRK29 having an anti-fibrosis effect is screened from a polypeptide library and subsequently optimized. In an early research stage, a team of the present invention knows that phosphorylation of the polypeptide can be inhibited by binding to a structural domain MH2 of a protein SMAD3, thereby blocking the activation of a

TGFb pathway and leading to inhibition of fibrosis. Therefore, several polypeptides having strong binding ability to the structural domain MH2 of the SMAD3 are first screened from a polypeptide library by the team of the present invention in the early research stage. As further found from the screened polypeptides, a polypeptide FK (having an amino acid sequence as shown in SEQ ID NO. 2) is most closely bound to the structural domain MH2 of the SMAD3. A specific process is as follows.

First, specific conditions were set by the team of the present invention according to previous research results to obtain a polypeptide library, then polypeptides were selected from the polypeptide library, and the polypeptides and the structural domain MH2 of human SMAD3 (PDB DOI: 10.2210/pdb1MJS/pdb) were subjected to computer-simulated docking by CABS-dock software to obtain an average RMSD value of an optimal docking mode of the polypeptides. The polypeptides were randomly selected for several times and subjected to the computer-simulated docking to obtain Top15 polypeptides having strong binding ability to the structural domain MH2 of the SMAD3 (as shown in FIG. 1 and Table 1). Further, it is found that the FK has the strongest binding ability to the structural domain MH2 of the SMAD3. The polypeptide FK has 20 amino acids. As found in previous research of the team of the present invention, polypeptides with an amino acid number of 10-40 usually have higher transformation significance; when the amino acid number is too small, a tertiary structure is difficult to form and is more unstable; and when the amino acid number is too large, the cell penetrability of polypeptides is reduced, and the synthesis cost is greatly increased. Therefore, the screened FK satisfies the above conditions.

Table 1: Sequences of Top15 polypeptides having strong binding ability to a structural 5 domain MH2 of SMAD3

| Polypeptide | Sequence |
|---|---|
| RK | FQGQYFHSRQYKHPVGYEGK |
| IW | IRYEHNTDAWAKKFFFTAYW |
| QM | QNTAHEWNSGDEPELIQMAM |
| DF | DCWWRKRHNCCEQILICENF |
| WR | WEQIHKVTCETQYKPQDPGR |
| PG | PHPPIWECCPVAEVEDVDIG |
| SR | SSGSRWFPGRMNSSPMEVVR |
| TA | TMYVYSLAPEWGSHWECVNA |
| YE | YTSYHDVHSHVSSAHYSWSE |
| FE | FNKMQFWAIYYKMVHPAVVE |
| GW | GMGHTYGHLFVSHQTGGMHW |
| CA | CDIMISEIASEMAWVHFVPA |
| HG | HGADINMVLWMIKRSNRPPG |
| LA | LIFCAERQAGWECSAYTDYA |
| ND | NWGALSWMNSEGWTVRRVID |

In order to further improve the cell penetrability of the polypeptide FK and enable the polypeptide to enter cells to achieve an effect of inhibiting a TGFb pathway, the amino acid sequence of the polypeptide FK is further optimized in the present invention. According to different optimization designs, two amino acid sequences of the polypeptide are preliminarily obtained, where the first amino acid sequence is as shown in SEQ ID NO. 1 (namely, the polypeptide PAFRK29), and the second amino acid sequence is FQGQYFHSRQYKHPVGYEGKRKKRRQRRR. According to computer-simulated binding analysis, it is finally confirmed that the polypeptide PAFRK29 is more closely bound to the structural domain MH2 of the SMAD3 (an average RMSD value of an optimal docking mode of the PAFRK29 and the structural domain MH2 of the SMAD3 is 0.847954, and an average RMSD value of the second polypeptide is 1.0328, where a smaller average RMSD value indicates closer binding), and excellent cell penetrability can be obtained. Therefore, the polypeptide PAFRK29is selected for further functional verification.

### Example 2: HPLC Purification Results and MS Identification Results of the Polypeptide PAFRK29

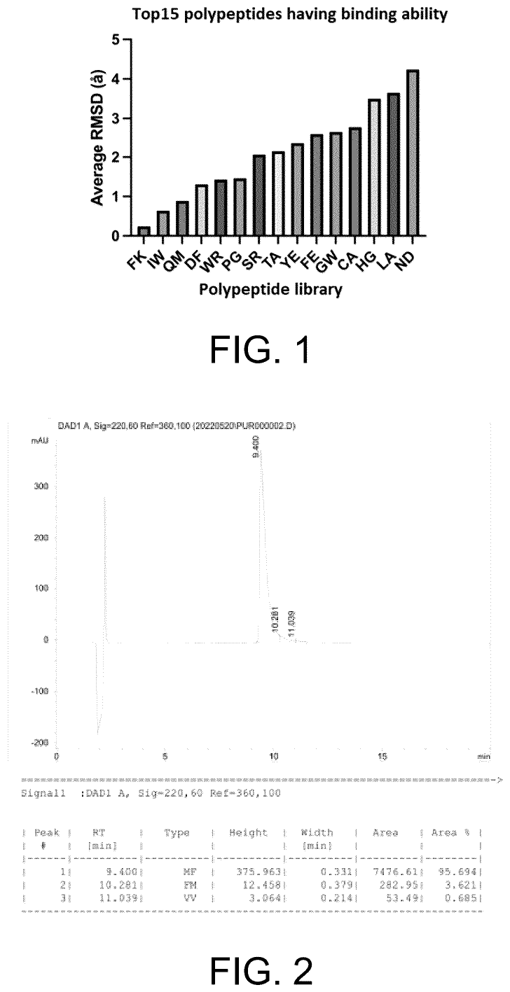

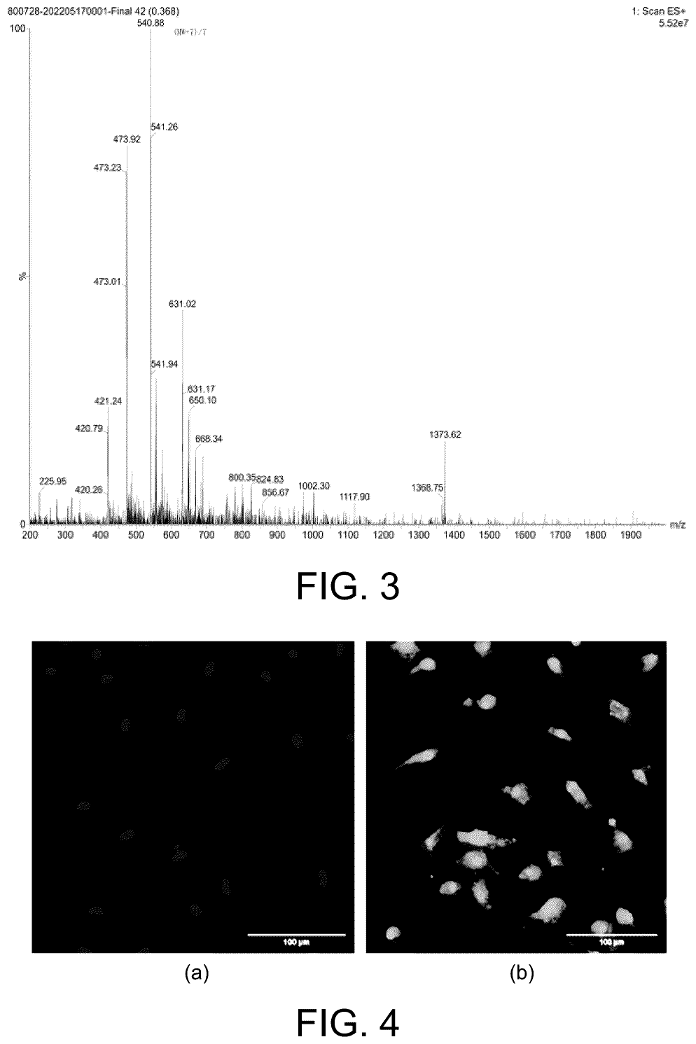

The polypeptide PAFRK29 was synthesized by a conventional polypeptide synthesis method in the art, and purified and identified by high performance liquid chromatography HPLC and mass spectrometry MS. Results are shown in FIG. 2 and FIG. 3, respectively. A sample is detected to have a purity of 95.694% after purification by high performance liquid chromatography HPLC, no errors are found after identification by mass spectrometry MS, and a molecular weight is 3778.33.

### Example 3: Evaluation of the Ability of the Polypeptide PAFRK29 to Penetrate Cell Membranes and Enter Target Cells

Primary neonatal rat cardiac fibroblasts (NRCF) were isolated, cultured in a high-glucose DMEM culture medium containing 10% of fetal bovine serum, and passed into a 24-well plate at 1:2 after the cells overgrew. When the cells overgrew to 70% confluency, the culture medium was changed into a serum-free low-glucose DMEM culture medium. Meanwhile, the polypeptide PAFRK29 with a fluorescent marker FITC was added into the low-glucose culture medium in an experimental group (a final concentration of the polypeptide PAFRK29 with the fluorescent marker FITC was 6 uM), only the low-glucose culture medium was added in a control group, and the two groups were co-cultured for 2 hours. A culture supernatant was discarded, washed with a PBS buffer for 2 times, fixed with 4% paraformaldehyde at room temperature for 10 minutes, and washed with a PBS buffer for 3 times. 250 ul of a PBS buffer containing a DAPI cell nucleus dye was added, and fluorescence of the FITC and the DAPI was observed under an inverted fluorescence microscope and photographed.

As shown in FIG. 4(a), only the morphology of positive cell nuclei with a blue DAPI cell nucleus dye can be observed in the control group. As shown in FIG. 4(b), in the group added with 6 uM of the polypeptide PAFRK29 with the fluorescent marker FITC, obvious aggregation of green FITC fluorescent signals in the neonatal rat cardiac fibroblasts can be observed. It is indicated that the polypeptide PAFRK29 is sufficient to penetrate cell membranes and enter target cells after co-culture for only 2 hours.

### Example 4: Evaluation of an Inhibitory Effect of the Polypeptide PAFRK29 on Expression of Fibrosis Markers and Extracellular Matrix Proteins

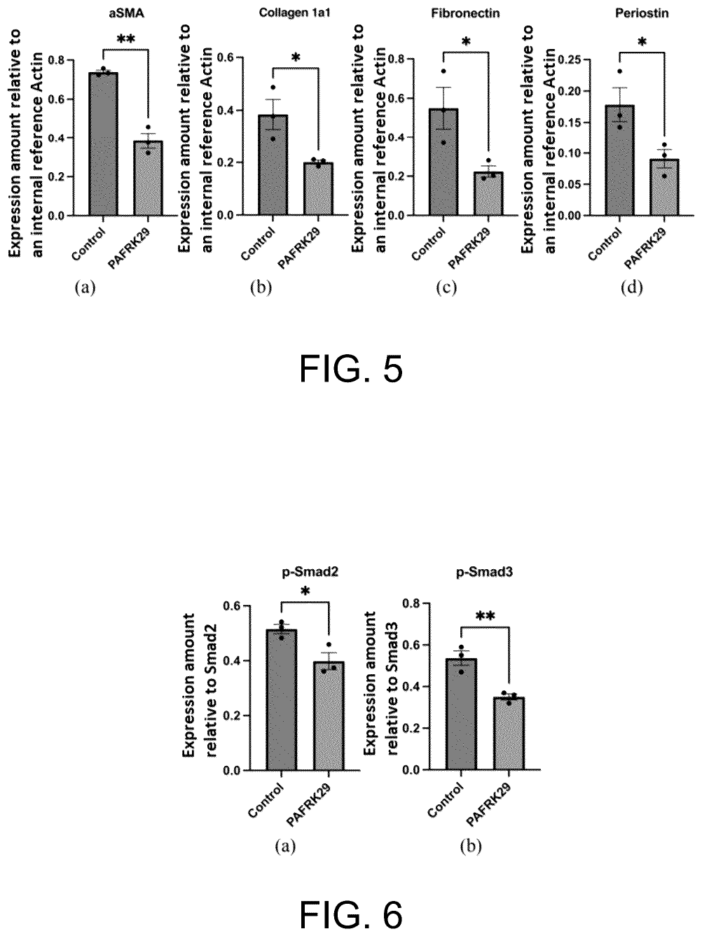

Primary neonatal rat cardiac fibroblasts (NRCF) were isolated, cultured in a high-glucose DMEM culture medium containing 10% of fetal bovine serum, and passed into a 12-well plate at 1:2 after the cells overgrew. When the cells overgrew to 70% confluency, the culture medium was changed into a serum-free low-glucose DMEM culture medium, and the cells were cultured overnight. Then, the polypeptide PAFRK29 and TGFb were added into the low-glucose culture medium (a final concentration of the polypeptide PAFRK29 was 6 uM, and a final concentration of the TGFb was 10 ng/ml), only the TGFb was added into the low-glucose culture medium (a final concentration of the TGFb was 10 ng/ml) in a control group, and the two groups were co-cultured for 48 h. A culture supernatant was discarded, washing was performed with a PBS buffer for 2 times, and an RIPA cell lysis buffer containing a protease and phosphatase inhibitor was added at 100 ul/well. Standing was performed on ice for 5 minutes. A protein was scraped with a cell scraper into a 1.5 ml EP tube, placed on ice for standing for 15 minutes, and then placed into a centrifuge for centrifugation at 12,000 g for 10 minutes. 90 ul of a supernatant was taken into a new EP tube after the centrifugation. Then, the protein concentration was determined by a BCA method. After the protein concentration was determined, the protein was diluted to a same concentration with a 2.5X loading buffer containing 2-mercaptoethanol. Then, the protein was boiled in a metal bath at 99° C. for 10 minutes. Then, Western blot protein electrophoresis and membrane transfer were performed. After the membrane transfer, a PVDF membrane was blocked with a PBST solution containing 5% of bovine serum albumin for 1 hour. Then, primary antibodies against aSMA, Periostin, Fibronectin and Collagen lal (antibody article numbers were ab5694, AF2955, ab2413 and ab270993, respectively) were prepared with a primary antibody diluent at a volume ratio of 1:1000 and incubated overnight at 4° C. The primary antibodies were recovered and washed with PBST for 5 times for 3 minutes each time. Then, formulated HRP conjugated secondary antibodies against species from which the corresponding primary antibodies were derived were added. Incubation was performed at room temperature for 1 hour. The secondary antibodies were discarded, and washing was performed with PBST for 5 times for 3 minutes each time. Then, strip development and imaging were performed with a developing solution. An internal control Actin was incubated with a direct labeled antibody HRP (diluted at 1:6000) at room temperature for 1 hour. The internal control Actin and the direct labeled antibody HRP were discarded, washing was performed with PBST for 5 times for 3 minutes each time, and then, strip development and imaging were performed with a developing solution. A gray value was calculated by Bio-Rad Image Lab Software 6.1. As shown in FIG. 5(a-d), in a polypeptide PAFRK29 treatment group, relative expression amounts of a fibrosis marker protein aSMA and extracellular matrix proteins, such as Periostin, Fibronectin and Collagen lal, are obviously decreased compared with that in the control group.

### Example 5: Evaluation of an Inhibitory Effect of the Polypeptide PAFRK29 on a Classic Signaling Pathway Smad2/3 of Fibrosis

Primary neonatal rat cardiac fibroblasts (NRCF) were isolated, cultured in a high-glucose DMEM culture medium containing 10% of fetal bovine serum, and passed into a 12-well plate at 1:2 after the cells overgrew. When the cells overgrew to 70% confluency, the culture medium was changed into a serum-free low-glucose DMEM culture medium, and the cells were cultured overnight. Then, the polypeptide PAFRK29 and TGFb were added into the low-glucose culture medium (a final concentration of the polypeptide PAFRK29 was 6 uM, and a final concentration of the TGFb was 10 ng/ml), only the TGFb was added into the low-glucose culture medium (a final concentration of the TGFb was 10 ng/ml) in a control group, and the two groups were co-cultured for 48 h. A culture supernatant was discarded, washing was performed with a PBS buffer for 2 times, and an RIPA cell lysis buffer containing a protease and phosphatase inhibitor was added at 100 ul/well. Standing was performed on ice for 5 minutes. A protein was scraped with a cell scraper into a 1.5 ml EP tube, placed on ice for standing for 15 minutes, and then placed into a centrifuge for centrifugation at 12,000 g for 10 minutes. 90 ul of a supernatant was taken into a new EP tube after the centrifugation. Then, the protein concentration was determined by a BCA method. After the protein concentration was determined, the protein was diluted to a same concentration with a 2.5x loading buffer containing 2-mercaptoethanol. Then, the protein was boiled in a metal bath at 99° C. for 10 minutes. Then, Western blot protein electrophoresis and membrane transfer were performed. After the membrane transfer, a PVDF membrane was blocked with a PBST solution containing 5% of bovine serum albumin for 1 hour. Then, primary antibodies against p-Smad2 (Ser465/467), Smad2, p-Smad3 (Ser423/425) and Smad3 (antibody article numbers were CST #5339, #3108, #9523 and #9520, respectively) were prepared with a primary antibody diluent at a volume ratio of 1:1000 and incubated overnight at 4° C. The primary antibodies were recovered and washed with PBST for 5 times for 3 minutes each time. Then, formulated HRP conjugated secondary antibodies against species from which the corresponding primary antibodies were derived were added. Incubation was performed at room temperature for 1 hour. The secondary antibodies were discarded, washing was performed with PBST for 5 times for 3 minutes each time, and then, strip development and imaging were performed with a developing solution. A gray value was calculated by Bio-Rad Image Lab Software 6.1.

As shown in FIG. 6(a-b), in a polypeptide PAFRK29 treatment group, a p-Smad2/Smad2 ratio and a p-Smad3/Smad3 ratio are obviously decreased compared with that in the control group, indicating that an SMAD2/3 pathway in the polypeptide PAFRK29 treatment group is obviously inhibited.

### Example 6: Evaluation of Retension Situation of the Polypeptide PAFRK29 in the Heart After Intramyocardial Injection

Healthy 8-week-old C57 mice were evenly divided into 4 groups with 3 mice in each group. On 1 hour, 3 days, 5 days and 7 days before sampling, the mice were administered once with the polypeptide PAFRK29 with a fluorescent marker Cy3 by intramyocardial injection, respectively. 1 ug of the polypeptide PAFRK29 with the fluorescent marker Cy3 was dissolved in 30 ul of physiological saline, 3 points were used, and 10 ul was injected into the myocardium of the left ventricular anterior wall of the heart at each point. Then, sampling was performed at the same time points. Fresh hearts of the mice were taken out and placed on a same 10 cm culture dish, and the intensity of Cy3 fluorescent signals was detected by a small animal living imager, where the heart of a mouse without the injection of Cy3 was used as a negative control. The Cy3 fluorescence intensity of the hearts of all other mice relative to the negative control was calculated and analyzed statistically.

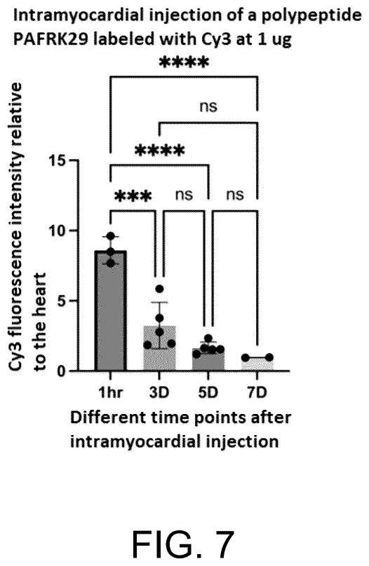

As shown in FIG. 7, on day 5 after intramyocardial injection of the polypeptide PAFRK29 with the fluorescent marker Cy3, some residual Cy3 fluorescent signals are still found in the heart, indicating that the polypeptide PAFRK29 can remain in the heart for a long time. Example 7: Evaluation of Cardiac Fibrosis and Cardiac Functions of Mice with Myocardial Infarction After Intramyocardial Injection of the Polypeptide PAFRK29

8-week-old C57 mice were selected for myocardial infarction modeling and then randomly divided into two groups with 7 mice in each group. On day 6 after the modeling, a cardiac systolic function was evaluated under ultrasonic conditions to ensure that the two groups have no difference in a cardiac baseline function. Then, on day 7, day 10, day 13, day 18 and day 23 after myocardial infarction, ultrasound-guided intramyocardial injection was performed for 5 times. 1ug of the polypeptide PAFRK29 was injected in the experimental group each time. 1 ug of the polypeptide PAFRK29 was dissolved in 30 ul of physiological saline, 3 points were used, and 10ul was injected into the myocardium of the left ventricular anterior wall of the heart at each point. 1 ug of a control polypeptide (with an amino acid sequence of RKKRRQRRR) was injected in the control group each time by a same method. 4 weeks after the modeling, the cardiac systolic function was evaluated under ultrasonic conditions, then sampling was performed, and the general hearts of the mice were photographed. The hearts were placed in a 30% sucrose solution for 1day, and then OCT embedding was performed. Then, frozen sections with a thickness of 6 um were made. Sectioning was performed from the apex of the heart, the sections were collected when annular tissue structures were found, one section was collected every 500 um, and 6 sections were collected for each heart. Then, staining was performed with Sirius Red, and the proportion of an infarction fibrosis area was calculated statistically by ImageJ software.

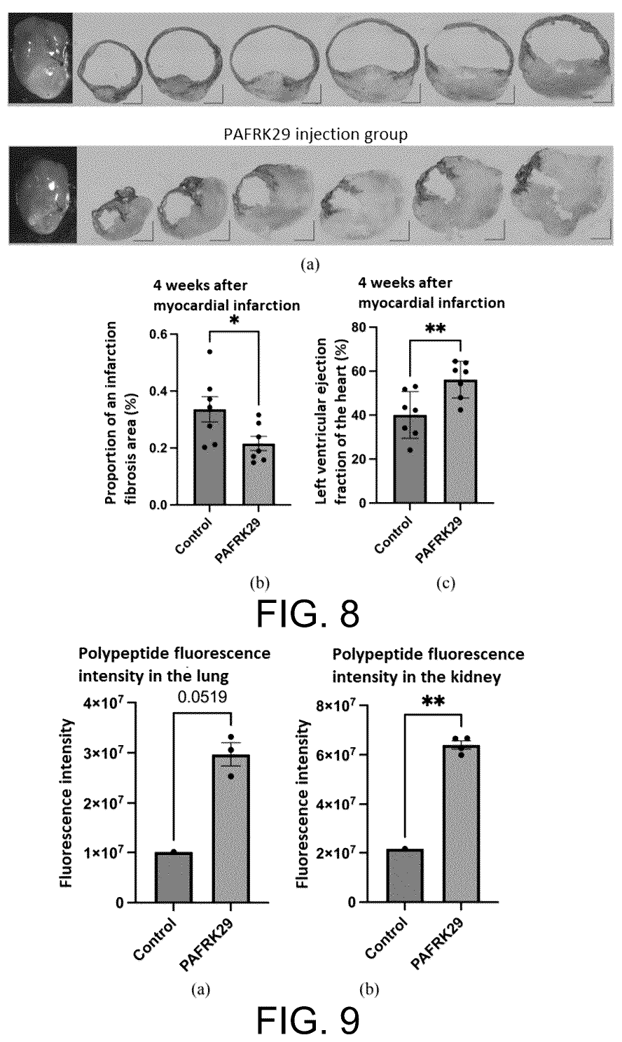

As shown in FIG. 8(a), representative general hearts and Sirius Red staining situations of various sections spaced at 500 um in the control group and the experimental group are shown. As can be seen, compared with the control group, the PAFRK29 injection group has a smaller infarction fibrosis area.

As shown in FIG. 8(b), a statistical chart of Sirius Red staining indicates that compared with the control group, the PAFRK29 injection group has a lower infarction fibrosis area proportion and has statistical differences.

As shown in FIG. 8 (c), 4 weeks after the myocardial infarction, compared with the control group, the PAFRK29 injection group has a higher left ventricular ejection fraction and has statistical differences, indicating that a better left ventricular cardiac function is achieved.

### Example 8: Evaluation of Distribution Situations of the Polypeptide PAFRK29 in the Lung and Kidney After Tail Vein Injection

On 2 hours before sampling, 4 healthy 8-week-old C57 mice were administered once with the polypeptide PAFRK29 with a fluorescent marker Cy3 by tail vein injection. The polypeptide PAFRK29 with the fluorescent marker Cy3 was dissolved in physiological saline and administered by tail vein injection at a dose of 10 mg/kg. Then, sampling was performed 2 hours after the injection. Lungs and kidneys of the mice were taken out and placed on two 10 cm culture dishes, respectively, and the intensity of Cy3 fluorescent signals was detected by a small animal living imager, where the heart of a mouse without the injection of Cy3 was used as a negative control.

The average Cy3 fluorescence intensity of the lungs and kidneys of all the mice was calculated and analyzed statistically.

As shown in FIG. 9(a-b), after tail vein injection of the polypeptide PAFRK29 with the fluorescent marker Cy3, Cy3 fluorescence signals are obviously distributed in both the lungs and kidneys, indicating that the polypeptide PAFRK29 can be enriched into the lungs and kidneys after the tail vein injection.

### Example 9: Evaluation of Renal Fibrosis of renal Fibrosis Modeling Mice After Tail Vein Injection of the Polypeptide PAFRK29

8-week-old C57 mice were selected for renal fibrosis modeling (with a unilateral ureteral obstruction model) on day 0, and then randomly divided into two groups with 8 mice in each group.

On day 1 after the modeling, tail vein injection was performed once every other day (for a total of 7 times). The polypeptide PAFRK29 was injected in the experimental group according to the body weight of the mice each time, and was injected at 10 mg/kg body weight. A control polypeptide (with an amino acid sequence of RKKRRQRRR) was injected in the control group by a same method according to the body weight of the mice each time, and was injected at 10mg/kg body weight. Sampling was performed 2 weeks after the molding. Kidneys were fixed in a 4% paraformaldehyde solution for 1 day, dehydrated and subjected to paraffin embedding. Then, paraffin sections with a thickness of 4 um were prepared. Then, Masson collagen staining was performed, a 200x high-power image of a glomerular region of each mouse was taken with a microscope, the proportion of a fibrosis region was calculated statistically by ImageJ software, and an average percentage of the fibrosis region of each mouse was calculated.

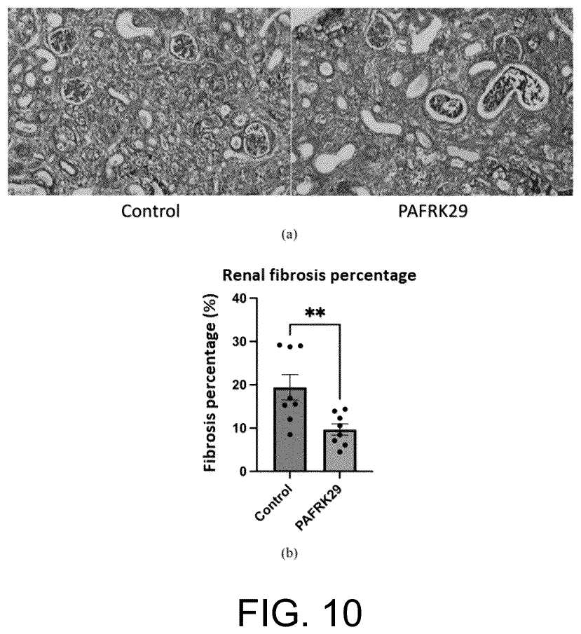

As shown in FIG. 10 (a), representative renal Masson collagen staining images of the control group and the experimental group are shown, where a blue area is a fibrosis collagen deposition area. As can be seen, compared with the control group, the PAFRK29 injection group has a smaller fibrosis area.

As shown in FIG. 10 (b), a statistical chart of the percentage of a Masson collagen staining area indicates that compared with the control group, the PAFRK29 injection group has a lower fibrosis area proportion and has statistical differences.

### Example 10: Evaluation of Pulmonary Fibrosis of Pulmonary Fibrosis Modeling Mice After Tail Vein Injection of the Polypeptide PAFRK29

8-week-old C57 mice were selected for pulmonary fibrosis modeling (bleomycin was administered for 1 time by intratracheal injection at a dose of 2 ug bleomycin/g mouse body weight) on day 0, and then randomly divided into two groups with 8 mice in each group. On day 1 after the modeling, tail vein injection was performed once every other day (for a total of 7 times). The polypeptide PAFRK29 was injected in the experimental group according to the body weight of the mice each time, and was injected at 10 mg/kg body weight. A control polypeptide (with an amino acid sequence of RKKRRQRRR) was injected in the control group by a same method according to the body weight of the mice each time, and was injected at 10 mg/kg body weight. Sampling was performed 2 weeks after the molding. Lungs were fixed in a 4% paraformaldehyde solution for 1 day, dehydrated and subjected to paraffin embedding. Then, paraffin sections with a thickness of 4 um were prepared. Then, Masson collagen staining was performed, a 200x high-power image of an alveolar region of each mouse was taken with a microscope, the proportion of a fibrosis region was calculated statistically by ImageJ software, and an average percentage of the fibrosis region of each mouse was calculated.

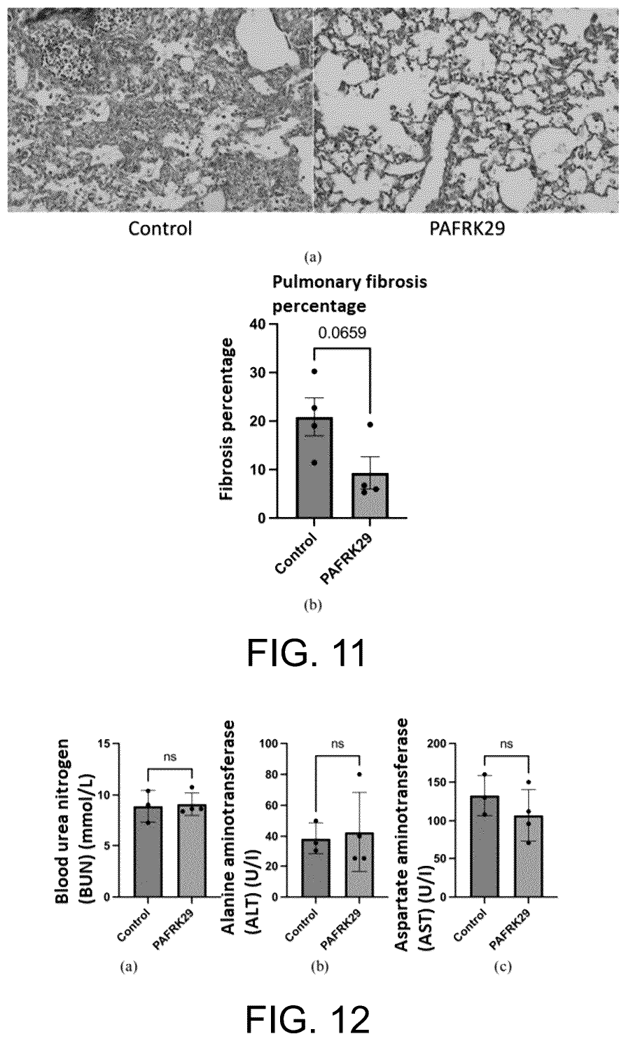

As shown in FIG. 11(a), representative pulmonary Masson collagen staining images of the control group and the experimental group are shown, where a blue area is a fibrosis collagen deposition area. As can be seen, compared with the control group, the PAFRK29 injection group has a smaller fibrosis area and a smaller pulmonary fibrosis consolidation range.

As shown in FIG. 11(b), a statistical chart of the percentage of a Masson collagen staining area indicates that compared with the control group, the PAFRK29 injection group has a lower fibrosis area proportion.

### Example 11: Evaluation of a Toxic or Side Effect of the Polypeptide PAFRK29 on Mice After Injection for Several Times

During sampling of the mice with myocardial infarction modeling for 4 weeks and intramyocardial injection of the polypeptide PAFRK29 for 5 times in Example 6, peripheral blood of the mice was collected into an anticoagulant tube and centrifuged at 1,000 g for 10 minutes. Serum of a supernatant was taken, and levels of blood urea nitrogen (BUN), serum alanine aminotransferase (ALT) and serum aspartate aminotransferase (AST) were determined by an automatic biochemical analyzer.

As shown in FIG. 12(a-c), compared with a control group, a polypeptide PAFRK29injection group has no obvious differences in the levels of blood urea nitrogen (BUN), serum alanine aminotransferase (ALT) and serum aspartate aminotransferase (AST) of the mice, which are all within a physiological range of normal C57 mice. It is indicated that the polypeptide PAFRK29 has no obvious toxic or side effect on the mice at an effective anti-fibrosis dose.

1. A polypeptide, comprising an amino acid sequence as shown in SEQ ID NO. 1.

2. Application of the polypeptide according to claim 1 in preparation of an anti-fibrosis drug.

3. The application according to claim 2, wherein the fibrosis is cardiac fibrosis, pulmonary fibrosis, renal fibrosis, skin fibrosis, liver cirrhosis, or myelofibrosis.

4. The application according to claim 3, wherein the cardiac fibrosis is cardiac fibrosis caused by ischemic heart disease, hypertensive heart disease, cardiomyopathy, arrhythmia, or rheumatic heart disease.

5. The application according to claim 2, wherein the anti-fibrosis drug comprises: the polypeptide as an main active ingredient and a pharmaceutically acceptable carrier and/or an excipient.

6. The application according to claim 2, wherein the anti-fibrosis drug is a pharmaceutical preparation administered by injection, oral administration, intranasal administration, pulmonary administration, rectal administration, oral mucosal administration, or skin administration.

7. The application according to claim 6, wherein the injection comprises intramyocardial injection, intradermal injection, subcutaneous injection, intramuscular injection, and intravenous injection.

8. An anti-fibrosis drug, comprising:
the polypeptide according to claim 1, and a pharmaceutically acceptable carrier and/or an excipient.

9. The anti-fibrosis drug according to claim 8, wherein the anti-fibrosis drug is a pharmaceutical preparation administered by injection, oral administration, intranasal administration, pulmonary administration, rectal administration, oral mucosal administration, or skin administration.

10. The anti-fibrosis drug according to claim 9, wherein the injection comprises intramyocardial injection, intradermal injection, subcutaneous injection, intramuscular injection, and intravenous injection.

11. The application according to claim 5, wherein the anti-fibrosis drug is a pharmaceutical preparation administered by injection, oral administration, intranasal administration, pulmonary administration, rectal administration, oral mucosal administration, or skin administration.

12. The application according to claim 11, wherein the injection comprises intramyocardial injection, intradermal injection, subcutaneous injection, intramuscular injection, and intravenous injection.


SEQUENCE LISTING (18 sequences)

SEQ ID NO: 1 - protein, 29 aa, synthetic construct
RKKRRQRRRFQGQYFHSRQYKHPVGYEGK

SEQ ID NO: 2 - protein, 20 aa, synthetic construct
FQGQYFHSRQYKHPVGYEGK

SEQ ID NO: 3 - protein, 20 aa, synthetic construct
IRYEHNTDAWAKKFFFTAYW

SEQ ID NO: 4 - protein, 20 aa, synthetic construct
QNTAHEWNSGDEPELIQMAM

SEQ ID NO: 5 - protein, 20 aa, synthetic construct
DCWWRKRHNCCEQILICENF

SEQ ID NO: 6 - protein, 20 aa, synthetic construct
WEQIHKVTCETQYKPQDPGR

SEQ ID NO: 7 - protein, 20 aa, synthetic construct
PHPPIWECCPVAEVEDVDIG

SEQ ID NO: 8 - protein, 20 aa, synthetic construct
SSGSRWFPGRMNSSPMEVVR

SEQ ID NO: 9 - protein, 20 aa, synthetic construct
TMYVYSLAPEWGSHWECVNA

SEQ ID NO: 10 - protein, 20 aa, synthetic construct
YTSYHDVHSHVSSAHYSWSE

SEQ ID NO: 11 - protein, 20 aa, synthetic construct
FNKMQFWAIYYKMVHPAVVE

SEQ ID NO: 12 - protein, 20 aa, synthetic construct
GMGHTYGHLFVSHQTGGMHW

SEQ ID NO: 13 - protein, 20 aa, synthetic construct
CDIMISEIASEMAWVHFVPA

SEQ ID NO: 14 - protein, 20 aa, synthetic construct
HGADINMVLWMIKRSNRPPG

SEQ ID NO: 15 - protein, 20 aa, synthetic construct
LIFCAERQAGWECSAYTDYA

SEQ ID NO: 16 - protein, 20 aa, synthetic construct
NWGALSWMNSEGWTVRRVID

SEQ ID NO: 17 - protein, 29 aa, synthetic construct
FQGQYFHSRQYKHPVGYEGKRKKRRQRRR

SEQ ID NO: 18 - protein, 9 aa, synthetic construct
RKKRRQRRR